(19) United States
(12) Patent Application Publication
(10) Pub. No.: US 2004/0213834 A1
(43) Pub. Date: Oct. 28, 2004

(54) Pharmacological preparation made from a nanopatriculate mesomorphous polyelectrolyte lipid complex and at least one active ingredient

(76) Inventors: Andreas Thuenemann, Potsdam (DE); Sascha General, Potsdam (DE)

Correspondence Address: D Edward Dolgonrkov, Marshall & Melhorn, 8th Floor, Four Seagate, Toledo, OH, 43604, US

(21) Appl. No.: 10/482,352

(22) PCT Filed: Jul. 2, 2002
(86) PCT No.: PCT/EP02/07255

(30) Foreign Application Priority Data
Jul. 5, 2001 (DE) 101-32-669.6

(51) Int. Cl. A61K 9/127

(52) U.S. Cl. 424/450

(57) ABSTRACT

The invention relates to a pharmacological preparation made from a nanoparticulate mesomorphous polyelectrolyte lipid complex and at least one active ingredient. The polyelectrolyte lipid complex has thereby a lamellar structure comprising alternate ionic and non-ionic layers, the active ingredient being incorporated in the non-ionic layer.

The invention relates to a pharmacological preparation made from a nanoparticulate mesomorphous polyelectrolyte lipid complex and at least one active ingredient. The polyelectrolyte lipid complex has thereby a lamellar structure comprising alternate ionic and non-ionic layers, the active ingredient being incorporated in the non-ionic layer.

Multi-charged macromolecular compounds form with ions of opposite charge ionic compounds which, dependent upon the charge distribution, the molecular weight and the hydrophobicity of the end product, are often precipitated from aqueous solutions. Lower molecular ions of equal charge are thereby displaced by the higher molecular compound. Known examples in this field are the formation of gels by adding together alginate solutions and Ca²⁺. Likewise, protein precipitations are implemented according to this principle. Polyelectrolyte lipid complexes can in principle be formed either from a macromolecular, multicharged component of one polarity and many lower molecular ions of the other polarity, or else be formed from two macromolecular, respectively multi-charged partners of different polarity. Pharmaceutical preparations are known from DE 40 13 110 A1 which contain polyelectrolyte lipid complexes in microparticulate form.

In modern pharmaceutical technology, formulations and active ingredient combinations are becoming more and more important, which provide not only the active ingredient in an applicable form but specifically influence the biodistribution, the bioavailability or the resorption of the medicament. As a result, the route to new therapeutic diagnostic applications fields is opened. At the same time, the therapeutic range of an active ingredient can also be improved by this route. In particular particulate systems of the smallest diameter (so-called micro- or nanoparticles) have proved in recent times to be an important application form both in the oral and in the parenteral sphere. (R. H. Mullet et al., “Pharmaceutical Technology: Modern Drug Forms”, Wissenschaftliche Verlagsgesellschaft mbH, 1997).

It is therefore the object of the present invention to provide a pharmacological preparation in a formulation which is easy to apply and makes it possible to control the biodistribution, bioavailability and resorption of the active ingredient.

This object is achieved by the pharmacological preparation having the features of claim 1. The further dependent claims denonstrate advantageous developments.

According to the invention, a pharmacological preparation made from a nanoparticulate mesomorphous polyelectrolyte lipid complex and at least one active ingredient is provided. There is understood by mesomorphous here an order state analogous to that of a liquid crystal which can be identified unequivocally by means of colloid-analytical methods, such as X-ray small angle scattering. The polyelectrolyte lipid complex has according to the invention a lamellar structure comprising alternate ionic and non-ionic layers, the at least one active ingredient being incorporated in the non-ionic layer. A slow decomplexing is then effected in vivo via solution equilibrium and charge interaction, which leads to dissolution of the mesomorphous complex by releasing the active ingredient. The release of the active ingredient can thereby be influenced via the pH value of the surrounding medium.

Surprisingly, it was able to be shown that these polyelectrolyte lipid complexes according to the invention have a very high chemical stability and a controllable release of the active ingredient in nanoparticulate form. As a result, a simple route for immobilisation of chemically sensitive medicaments arises. Furthermore, the crystallisation of medicaments by this means of formulation can be suppressed in a simple manner.

In a preferred embodiment, the particulate polyelectrolyte lipid complex is formed from spherical ionic and non-ionic layers. The individual layers alternate hereby analogously to an onion skin structure.

In a further variant, the particle is formed from planar ionic and non-ionic layers. The individual layers hereby alternate in the form of degrees of latitude or degrees of longitude in the particle.

In a further preferred embodiment, the particle has a shell formed from a polymer which surrounds the particle. This shell formed from a polymer can be selected preferably from polyethylene oxide, polyamino acid, polyethylene imine, poly(diallyldimethylammonium chloride), poly(N-methyl-4-vinylpyridinium-chloride), poly(N-ethyl-4-vinylpyridinium chloride), poly(N-butyl-4-vinylpyridinium chloride), poly(4-vinyl-1-(3-sulfopropylpyridiniumbetaine), poly(4-vinyl-1-carboxymethylpyridiniumbetaine).

A further possibility for controlling the release of the at least one active ingredient is effected via the particle size. It is made possible consequently that the duration of the decomposition in the body is influenced by means of the size of the particle. The particle size is thereby preferably between 10 and 500 nm, particularly preferred between 100 and 300 nm.

For the complex formation of the polyelectrolyte lipid complex, biocompatible and biodegradable polybases, which occur naturally or are formed from natural units, are used for preference. The respective counterions comprise natural or synthetic lipids. There are thereby used preferably as lipids soya lecithin, egg lecithin, saturated or unsaturated fatty acids and/or salts thereof, e.g. dodecanoic-acid and/or azelaic-acid. There are used preferably as polybases polyethylene imine (PEI), derivatives thereof, polyamino-acids and/or chitosan. Likewise it is however also possible that block copolymers are used as polybases. There are included herein for example a block copolymer comprising a polyethylene oxide block and a polyamino-acid block or a block copolymer comprising a polyethylene oxide block and a polyethyleneimine block. The homopolymers of arginine, of histidine or of lysine are used as particularly preferred polyamino-acid blocks.

A particularly preferred polyelectrolyte lipid complex is for example a complex of polyethyleneimine and dodecanoic-acid and/or azelaic-acid and also a complex of a block copolymer comprising polyethylene oxide and polyethyleneimine and also dodecanoic-acid and/or azelaic-acid.

All the current compounds from pharmacology are possible as incorporated active ingredients. There are included therein for example active peptides, proteins, enzymes, enzyme inhibitors, antigens, cytostatics and/or antibiotics.

In a further variant, the particle surface can be chemically modified. There are included herein for example the coupling with antibodies or DNA.

The subject according to the invention is intended to be explained in more detail by means of the following example, without restricting the latter to these examples.

### EXAMPLE 1

Polyethyleneimine-Dodecanoic-Acid-Complexes

0.88 g (22 mmol) of PEI (M_{w}=25000 g/mol) were dissolved in 15 ml of a mixture of 4 parts ethanol and one part water. During agitation and at 65° C., 11 mmol of dodecanoic-acid (C12), dissolved in ethanol (65° C.), were added over a period of 15 min. After further 30 min agitation, the solution was dried in a Teflon dish at 40° C. to form a clear film. The detection of complete complexing of the carboxylic-acid functions was implemented with FT-IR. The disappearance of the typical IR carbonyl band at 1690 cm⁻¹ in the complex indicated the absence of free acid groups. The optical anisotropy of the complex was able to be detected with polarisation-microscopic pictures. The formed textures were identified as fan textures, typical for lamellar liquid crystals. DSC measurements showed a melting transition of the last 1.6 methylene groups of the alkyl side chains of dodecanoic-acid in the complex at 4° C. The absence of crystals in the complex at room temperature was able to be confirmed by means of X-ray wide angle scattering. Only an amorphous arrangement of the alkyl chains with an average spacing of the chains of 0.45 nm was found. The mesomorphous structure was examined with X-ray small angle scattering. A lamellar arrangement of the polyelectrolyte and of the ionic head group of the dodecanoic-acid alternating with the alkyl radicals of carboxylic-acid was detected. The lamellar spacing was thereby 2.9 nm and the stack order of the structure over 600 nm perpendicular to the lamellar normals.

The model medicaments coenzyme Q₁₀ and triiodothyronine were dissolved in ethanol or DMSO. Respectively 17.7 mg (triiodothyronine) and 25 mg (Q₁₀) were added to a solution of 100 mg of the complex in THF. The mixtures were dried in a Teflon dish. The absence of crystalline regions was able to be detected in turn by wide angle X-ray scattering and DSC measurements. Even after storing the laden complex for more than 150 days in room conditions, no crystalline reflexes were able to be found. The melting point of the methylene groups of the alkyl radicals dropped to −8° C. due to the loading with medicament, which indicates incorporation of the substances in this region of the lamella. A phase separation of complex and medicaments was able to be ruled out. Small angle X-ray scatterings indicated an increase in the lamellar spacing of approximately 0.3 nm due to the incorporation.

For the preparation of the nanoparticles, 20 mg of the complexes laden with 20% [w/w] Q₁₀ or 15% [w/w] triiodothyronine were dissolved in 15 ml THF. Subsequently, an addition of 20 ml water is effected over a period of 10 min. The THF was evaporated over 12 hours at 50° C. and during gentle agitation. Subsequently, the jointly evaporated water was supplemented again and the dispersions were filtered through an 800 nm membrane filter. By means of NMR, no THF was able to be detected in the dispersions. The detection limit of this method was thereby below 0.5% [w/w]. The nanoparticles showed a zeta potential of +50 mV and a size which was in the range of 100 to 200 nm corresponding to the starting concentration of the complex in the THF solution. The smallest particles were thereby obtained from a 0.1% solution (described here), the largest particles from a 1% original solution. By means of the zeta potential measurements, AFM pictures and TEM pictures, it was able to be shown that the particles have a nucleus-shell morphology. The nucleus is thereby formed from an almost stoichiometric complex of the PEI and of the dodecanoic-acid, the shell comprising non-complexed PEI chains; stoichiometry in batch 2 (PEI): 1 (dodecanoic-acid). The non-water-soluble model medicaments could be dispersed in these particles in water in a stable manner. No precipitate was found. By incorporating the fluorescence probe pyrene (likewise via the above-described route), a determination of the polarity of the surroundings of the pyrene, and hence of the surroundings of the incorporated medicaments, was able to be implemented. The surroundings were shown thereby to be rather hydrophobic, comparable to a solution of the pyrene in butanol. Such an environment is to be expected only within the complex and within the alkyl chain layer. The polarity within the complex is increased in the particles in comparison to the water-free complex film (comparable there to a hexane solution of the pyrene). By means of X-ray small angle scatterings, a mesomorphous structure within the particles was able to be detected. If one assumes a lamellar structure also in the particles, an increase in the lamellar spacing by the formation of nanoparticles to 4.0 nm is produced. Together with the determination of the micropolarity within the particles, the increase in the lamellar spacing can be explained as incorporation of water in the ionic layers of the complex. In comparison to the mesomorphous structure in unladen particles from the complex, the lamellar spacing is increased by the same 0.3 nm, as it was increased by the incorporation of the Q₁₀ in the complex film.

The particles were tested with respect to their stability against the influence of extraneous salt, dilution and pH value changes. The particle sizes of the particles were tested by means of DLS whilst changing the sodium chloride concentration of the aqueous medium. It was thereby shown that the particles were stable in size up to a NaCl concentration of 0.3 mol/l over a few days. Upon dilution of the particle dispersion, it was detected by online determination of the surface tension, conductivity and clouding that the particles remained stable in size and composition up to a concentration of 1 mg/l. Upon changes in the pH value of the aqueous medium, it was found that the particles did not remain stable below a pH value of 4.2 and above pH 8. A precipitate occurred below a pH value of 4.2 which was identified by means of the DSC as pure dodecanoic-acid. No particles were able to be detected at this pH value, i.e. the complex, which formed the nucleus of the particles, had separated, the dodecanoic-acid, which is water-insoluble at this pH value, having been precipitated and the PEI having remained molecular in solution. Upon increasing the pH value above pH 8, likewise no more particles were able to be observed. No precipitate was found. The complex like-wise separated, the residual components being water-soluble at this pH value. The conclusions were confirmed by tests of the polarity of the surroundings by the incorporation of the fluorescence probe pyrene. In the stability window of the particles between pH 4.2 and pH 8, the polarity was comparable to a butanol solution of the pyrene. Upon exceeding the values, the surroundings of the pyrene became more polar and reached the values for an aqueous solution of the pyrene.

### EXAMPLE 2

Polyamino-Acids-Dodecanoic-Acid-Complexes

For particle preparation, 40 ml of a 2 mmol/l (relative to the loads) solution of the polyamino-acids poly-L-histidine (PLH), poly-L-arginine (PLA) and poly-L-lysine (PLL) were placed in water and mixed slowly and during agitation with 4 ml of a 10 mmol/l (set at pH 9.5) aqueous solution of the dodecanoic-acid. Subsequently, the dispersion was filtered through an 800 nm membrane filter.

In order to incorporate the Q₁₀ and the pyrene, 3.4 mg or 0.3 mg were dissolved in 8 ml THF and mixed with the dodecanoic-acid solution before complexing. Subsequently, the THF was evaporated at 50° C. over a period of 12 hours. Here also, no traces of the solvent were able to be detected by means of NMR after evaporation of the THF. The particle sizes were determined via DLS for all polyamino-acids up to 180-205 nm. The zeta potential of the particles was dependent upon the used polyamino-acid+42 mV (PLH-dodecanoic-acid), +59 mV (PLL-C12) and +67 mV (PLA-C12). Together with AFM pictures of the particles, a nucleus-shell morphology was able to be detected. The nucleus comprises an almost stoichiometric complex, the shell comprises non-complexed polyamino-acid chains. Via CD measurements, it was able to be shown that the polyamino-acid in the complex, i.e. in the nucleus of the particles, is present in an ordered secondary structure, whereas the stabilising non-complexed chains on the surface of the particles occur in an unordered coil conformation. For the PLL and PLA dodecanoic-acid complexes within the particles, a domineering a-helix conformation was able to be detected, for the PLH dodecanoic-acid complex a β-pleated sheet conformation. By means of analytical ultracentrifugation, it was shown that incorporated Q₁₀ sediments with the same sedimentation coefficient as the nanoparticles, which permits the conclusion that the Q₁₀ is dispersed in the particles. Also after storing of the aqueous dispersion of the laden particles for more than 20 days, no precipitate of the water-insoluble Q₁₀ was able to be found. The particle sizes of the particles were tested by means of DLS whilst changing the sodium chloride concentration of the aqueous medium. It was thereby shown that the particles were stable in size up to an NaCl concentration of at least 0.3 mol/l for a few days. The stability of the particles was tested with respect to the zeta potential and particle size whilst changing the pH value of the aqueous medium. Corresponding to the pKs values of the polyamino-acids, the result with basic pH values was a drop in the zeta potentials and hence destabilisation of the particles and precipitation. This occurred for the PLH-C12 particles at a pH value of greater than 6.8, for the PLL-C12 particles at a pH value of greater than 9.8 and for the PLA-C12 particles at a pH value of greater than 11. Hence the particle destabilisation and complex dissolution is adjustable via the basicity of the polyamino-acids. With a pH value of less than 4.3, the result with all the polyamino-acids was separation of the complex and precipitation of the pure dodecanoic-acid, the polyamino-acids having remained in solution. The complex dissolution was tested by the fluorescence probe pyrene. In the respective stability windows of the complex particles, the polarity of the surroundings of the incorporated pyrene is rather nonpolar, upon exceeding the stability limits, the polarity approaches the values for aqueous surroundings of the pyrene, the pyrene is released into the aqueous medium.

### EXAMPLE 3

PEO-Block-PEI-Dodecanoic-Acid-Complexes

Respectively 100 mg of the 3 different polyelectrolyte block copolymers (correspond to 1.14*10⁻⁴ mol amine functions of the PEO-b-PEI_{cy}, 1.95*10⁻⁴ mol amine functions of the PEO-b-PEIₗᵢ and 2.8*10⁻⁴ mol amine functions of the PEO-b-PEI_{br}) were dissolved in 60° C. hot ethanol. Subsequently, 0.5 equivalents of dodecanoic-acid were added as 1% solution in hot ethanol. The clear solution was agitated for a further 30 min and subsequently poured into a Teflon dish and dried. By the addition of the corresponding quantities of Q₁₀ and triiodothyronine as solution in THF or DMSO to the solution of dodecanoic-acid before complexing, the incorporation of the model medicaments was achieved. The laden complexes were subsequently dried. A macroscopic orientation of the PEO-PEIₗᵢ-C12-complex was achieved by storage between two Teflon films under a mechanical pressure of 10 N/cm² over 12 hours. By FT-IR measurements, a complete complexing of the dodecanoic-acid was able to be detected. DSC measurement and X-ray wide angle scatterings showed that the crystallisation of the PEO chains was not impeded by complexing of the PEI block and that the PEI-C12 complex was present in an amorphous form. The incorporated model medicaments were thereby dispersed amorphously in the alkyl layer of the dodecanoic-acid. A structure hierarchy was detected by X-ray small angle scattering. A lamella of approximately 15 nm thickness is thereby produced by the block copolymer and a lamella, standing perpendicularly thereto, by the complex of the PEI blocks with the dodecanoic-acid. The complex lamella is with approximately 3 nm lamellar spacing 5 times smaller than the large lamella. The nanoparticles were prepared by controlled addition by dissolving by 20 ml water to 20 mg of the corresponding complex. After 30 minute agitation at 35° C., the dispersion was filtered through an 800 nm membrane filter. The zeta potential of the thus formed particles was 0 mV. The particle size, determined by DLS, was 200 nm. TEM pictures showed a nucleus-shell morphology of the particles, the nuclei comprising complexes of the PEI blocks and of the dodecanoic-acid and the shell comprising PEO chains. The particle morphology was able to be changed by varying the PEI block architecture from highly elongated via elongated to spherical.

1-14. (canceled).

15. A pharmacological preparation made from a nanoparticulate mesomorphous polyelectrolyte lipid complex and at least one active ingredient, the polyelectrolyte lipid complex having a lamellar structure comprising alternate ionic and non-ionic layers; wherein the at least one active ingredient is incorporated in the non-ionic layer, and the release thereof is controllable dependent upon the pH.

16. The preparation according to claim 15, wherein the particle is formed from spherical ionic and non-ionic layers.

17. The preparation according to claim 15, wherein the particle is formed from planar ionic and non-ionic layers.

18. The preparation according to claim 15, wherein the particle is surrounded by a shell formed from a polymer.

19. The preparation according to claim 18, wherein the shell is formed from at least one of polyethylene oxide, polyamino-acid, polyethyleneimine, poly(diallyldimethylammonium-chloride), poly(N-methyl-4-vinylpyridinium-chloride), poly(N-ethyl-4-vinylpyridinium-chloride), poly(N-butyl-4-vinylpyridinium-chloride), poly(4-vinyl-1-(3-sulfopropylpyridiniumbetaine), poly(4-vinyl-1-carboxymethylpyridiniumbetaine).

20. The preparation according to claim 15, wherein the release of the at least one active ingredient is controllable via the particle size.

21. The preparation according to claim 15, wherein the particle size is betweeen 10 and 500 nm.

22. The preparation according to claim 15, wherein the polyelectrolyte lipid complex is formed using soya lecithin, egg lecithin, saturated or unsaturated fatty acids and/or salts thereof as the lipid.

23. The preparation according to claim 15, wherein the polyelectrolyte lipid complex is formed using polyethyleneimine (PEI), derivatives thereof, a polyamino-acid and/or chitosan as the polybase.

24. The preparation according to claim 15, wherein the polyelectrolyte lipid complex is formed using block copolymers as the polybase.

25. The preparation according to claim 15, wherein the polyelectrolyte lipid complex is formed from polyethyleneimine and dodecanoic-acid and/or azelaic-acid.

26. The preparation according to claim 15, wherein the polyelectrolyte lipid complex is formed from a block copolymer comprising polyethylene oxide and polyethyleneimine and also dodecanoic-acid and/or azelaic-acid.

27. The preparation according to claim 15, wherein the at least one active ingredient is selected from the group consisting of: active peptides, proteins, enzymes, enzyme inhibitors, antigens, cytostatics and/or antibiotics.

28. The preparation according to claim 15, wherein the particle surface is chemical modified.

29. The preparation according to claim 15, wherein the particle size is between 100 and 300 nm.

30. The preparation according to claim 24, wherein the block polymers are from a polyethylene oxide block and a polyamino acid block, or from a polyethylene oxide block and a polyethyleneimine block as the polybase.

31. The preparation according to claim 28, wherein the chemical modification of the particles surface is accomplished by coupling with anti-bodies or DNA.
